(19) United States
(12) Patent Application Publication
(10) Pub. No.: US 2025/0073135 A1
(43) Pub. Date: Mar. 6, 2025

(54) DIFFRACTIVE EFFECT PIGMENTS HAVING A REFLECTIVE CORE AND SEMICONDUCTOR COATINGS

(71) Applicant: ECKART America Corporation, Painesville, OH (US)

(72) Inventor: Devin Schmitt, Munster, IN (US)

(21) Appl. No.: 18/720,794

(22) PCT Filed: Dec. 6, 2022
(86) PCT No.: PCT/IB2022/061829

§ 371 Date: Jun. 17, 2024

(30) Foreign Application Priority Data
Dec. 22, 2021 (EP) 21216797.7

(51) Int. Cl. A61K 8/02; C09D 11/02

(52) U.S. Cl. CPC A61K 8/0262; C09D 11/02; A61K 2800/437; A61K 2800/56; A61K 2800/651; A61K 2800/805

(57) ABSTRACT

Disclosed herein is a flaky diffractive effect pigment having a diffractive structure and comprising a flake of a highly reflective material having a first major interface and opposed to this first interface a second major interface, and at least one side surface and directly adjacent on one or of both of these major interfaces a layer of a semiconducting material having a bandgap of 0.7 to 2.5 eV. The diffractive effect pigment may be further coated with a coating which is optically non-active in the visible wavelength region.

The present invention relates to diffractive effect pigments having a reflective core and semiconductor coating layers. In general, diffractive effect pigments can be described as flakes of platy structures having a diffractive structure such as an embossing structure that show besides high reflection and gloss a diffraction effect which can comprise for example a whole or part of a whole rainbow spectrum.

Diffractive effect pigments are well known in the art and can generally be classified based on the core material for the flake of platy structure, which can be a metal or non-metal. Normally, this core material is coated with a number of different layers to provide for the desired optical effect.

Single-layered metallic diffractive effect pigments and methods of producing them are disclosed in U.S. Pat. Nos. 5,672,410 A, 5,629,068 A, 5,624,076 A, 6,068,691 A, and WO 1993/023481 A1.

US 2007/0207099 A1 discloses cosmetic applications, especially nail varnishes which are pigmented with diffractive single-layered metal pigments.

WO 2019/077021 A1 discloses leafing diffractive effect pigments and nail varnishes based on hydrocarbon binders containing these essentially single-layered effect pigments.

With such essentially single layered diffractive effect pigments fairly thin effect pigments with high hiding powder are feasible. Sometimes the diffraction structure of such thin metal pigments is mechanically not stable enough. US 2003/031870 A1 discloses diffractive effect pigments with one central metal layer which is mechanically stabilized on both sides with a dielectric layer. It also discloses a basic formula to calculate the diffraction effects with respect to the period length of the diffraction pattern. The thicknesses of the dielectric layers are rather high (at least 200 nm) leading to an effect pigment with rigidity, high mechanical strength but low hiding power.

Especially in security printing applications, however, a need exists for optical effects of higher complexity. Therefore, many patents disclose a diffractive effect pigment having interference structures. The interference structures are usually based on multilayer technology including Fabry-Perot-devices. Here stacks of absorbing, reflective and dielectric are formed in various manner using PVD technology. As dielectric layers typically metal oxides and or half-metal oxides with optical transparency and with a refractive index below 1.8 are used, such as SiO₂, Al₂O₃ and the like. Other materials with such optical properties are typically MgF₂, Al₂F₃, cerium fluoride, lanthanum fluoride, neodymium fluoride, samarium fluoride, barium fluoride, calcium fluoride, lithium fluoride and the like.

In these effect pigments the interference effects overlap with the diffractive effect which leads to very unusual color effect. Such effect pigments can, for example, exhibit a main strong reflection color combined with a rainbow effect, but muting certain ranges of the optical spectrum. However, with increasing number of layers the hiding power of the effect pigment decreases, because usually most of the pigment material is made from dielectrics. Furthermore, as the effect pigments have high total thicknesses and therefore rather low aspect ratios a misalignment of the pigments in the application media may occur in the sense that the plane-parallel orientation of the effect pigments is mute.

U.S. Pat. No. 6,692,830 B2 discloses diffractive effect pigments based on a central metal layer covered on both sides with dielectric layers and various variants thereof and also disclosing a basic formula to calculate the diffraction effects with respect to the period length of the diffraction pattern.

Various effect pigments for security articles are disclosed in WO 2001/53113 including multi-layered diffractive effect pigments.

US 2004/081807 A1 discloses security articles comprising diffractive effect pigments based on light transmissive substrate coated with an absorber layer, a dielectric layer overlying the absorber layer; and a reflector layer overlying the dielectric layer.

US 2003/190473 A1 discloses a diffractive pigment flake, comprising: a central reflector layer having a first major surface, an opposing second major surface, and at least one side surface; a first dielectric layer overlying the first major surface of the reflector layer; and a second dielectric layer overlying the second major surface of the reflector layer; wherein the first and second dielectric layers comprise a dielectric material having a refractive index of greater than about 1.3, and the pigment flake has a diffraction grating pattern throughout the layers thereof with at least about 1,400 grating lines per mm and a grating depth of at least about 100 nm. US 2003/104206 discloses various diffractive compositions of similar types of diffractive effect pigments.

Other such structures are disclosed in US 2006/077496 A1.

An achromatic diffractive pigment has been disclosed in EP 1463631 B1.

WO 2012/013568 A1 discloses diffractive effect pigments made by reactive vapor deposition process.

There is a need for diffractive effect pigments having a relative low thickness and appealing optical properties like a strong color flop or rainbow effect, possibly with omitting or muting selected colors and high gloss combined with high hiding power and a simple structure.

Another object is to provide a process to manufacture such effect pigment.

The present invention relates to a thin diffractive effect pigment that has a rather simple structure that shows some very favourable optical properties. The object is solved by providing a flaky diffractive effect pigment having a diffractive structure and a 3-layer stack consisting of a core of a flake of a highly reflective material having a first major interface and opposed to this first interface a second major interface, and at least one side surface and directly adjacent on both of these major interfaces a layer of a semiconducting material having a bandgap of 0.1 to 2.5 eV.

Further preferred embodiments are disclosed in claims 2 to 13.

A further object of the invention was solved by providing a method of manufacturing the diffractive effect pigment using a PVD process comprising the steps:
(a1) applying a release layer to a linearly movable flexible substrate,
(a2) introducing a diffractive structure into the release layer, preferably by embossing, and
(a3) depositing a first semiconductor layer onto the release layer on the linearly movable flexible substrate, in a vacuum chamber having a vapor-deposition section, by means of physical vapor deposition (PVD), and
(a4) depositing a metal layer onto the first semiconductor layer on the linearly movable flexible substrate, in a vacuum chamber having a vapor-deposition section, by means of physical vapor deposition
(a5) depositing a second semiconductor layer onto the metal layer on the linearly movable flexible substrate, in a vacuum chamber having a vapor-deposition section, by means of physical vapor deposition
   or
(b1) applying a release layer to a linearly movable flexible substrate,
(b2) depositing a first semiconductor layer onto the release layer on the linearly movable flexible substrate, in a vacuum chamber having a vapor-deposition section, by means of physical vapor deposition (PVD), and
(b3) depositing a metal layer onto the first semiconductor layer on the linearly movable flexible substrate, in a vacuum chamber having a vapor-deposition section, by means of physical vapor deposition
(b4) depositing a second semiconductor layer onto the metal layer on the linearly movable flexible substrate, in a vacuum chamber having a vapor-deposition section, by means of physical vapor deposition
(b5) introducing a diffractive structure onto and/or into any of the structures of step (b2) to (b4), preferably by embossing,
c) stripping the material stack from the flexible substrate in a solvent and
d) optionally further steps including particle sizing, particle classification and solvent exchange and/or solvent dispersion.

Further preferred embodiments of this process are disclosed in claims 15 to 16.

### Diffractive Effect Pigment

This invention is directed to a flaky diffractive effect pigment having a diffractive structure and a 3-layer stack consisting of a core of a flake of a highly reflective material having a first major interface and opposed to this first interface a second major interface, and at least one side surface and directly adjacent on both of these major interfaces a layer of a semiconducting material having a bandgap of 0.1 to 2.5 eV.

As this effect pigment is preferably made by a PVD process the semiconductor layers are essentially not coating the side surfaces of the core of a highly reflective material.

In preferred embodiments the diffractive effect pigment has a diffractive structure and a 3-layer stack consisting of a core of a flake of a highly reflective material having a first major interface and opposed to this first interface a second major interface, and at least one side surface and directly adjacent on both of these major interfaces a layer of a semiconducting material having a bandgap of 0.1 to 2.5 eV, wherein the semiconductor material is independently selected from semiconductor materials having an average atomic composition:
a. Si₍₁₋ₓ₎Geₓ, wherein 0≤x≤1.00
b. Si_{(1-y)}Sn_{y}, wherein 0<y<0.90
c. Ge_{(1-z)}Sn_{z}, wherein 0<z≤0.80
d. Si₍₁₋ₘ₋ₙ₎GeₘSnₙ, wherein 0<m<1.00, 0<n<1.00 and with the proviso that m+n<1.00 or
e. SiOₚ, wherein 0.30≤p≤1.50.

The x, y, z, n, m and p are mole fractions. In further preferred embodiments the single platelet semiconductor according to a) have compositions characterized by 0.02<x<0.9, more preferably 0.04≤x≤0.8 and most preferably 0.10≤x≤0.65. These materials are alloys of silicon and germanium. Germanium adds interesting color effects as this material is more absorbing in the visible wavelength region than silicon. Due to the high costs of this material the content of germanium is preferably as low as possible.

In a preferred embodiment the semiconductor layers are pure silicon layers (x=0). Most preferred is additionally an aluminum core coated with silicon on both sides (Si/Al/Si). These materials are rather inexpensive and non-toxic. They are especially usable for any kind of cosmetic applications.

In one embodiment the layers are of pure germanium (x=1). Such diffractive effect pigments can be preferably used in high price applications like security printing.

In further preferred embodiments the semiconductor materials used for coating layers of the two major interfaces of the metallic flake according to b) have compositions characterized by 0.02≤y≤0.75 and more preferred of 0.05≤y≤0.55. These materials are alloys of silicon and tin. They are especially advantageous for prismatic effect pigments when the zero-order reflectance color is intended to be in the bluish or greenish color region. With these silicon-tin alloys especially effect pigments having a zero-order reflection color in the bluish or greenish region are easy to achieve.

In further preferred embodiments the semiconductor materials used for coating layers of the two major interfaces of the metallic flake according to c) have compositions characterized by 0.02≤z≤0.5 and more preferred 0.05≤z≤0.4. These materials are alloys of germanium and tin, and they are also especially favorable for pigments with a reflection color in the bluish or greenish region.

In further preferred embodiments the semiconductor materials used for coating the two major interfaces of the metallic flake according to d) have compositions characterized by 0.02≤m≤0.8 and 0.02≤n≤0.75 and more preferred compositions characterized by 0.05≤m≤0.65, 0.05≤n≤0.55. These materials are alloys of silicon, germanium and tin and they are also especially favorable for pigments with a reflection color in the bluish or greenish region.

The molar amounts of the metals are preferably detected by ICP (Inductively Coupled Plasma combined with optical emission spectroscopy) after dissolving the whole effect pigments in acidic solutions.

In further preferred embodiments the semiconductor materials used for coating the two major interfaces of the metallic flake according to e) have compositions characterized by 0.50≤p≤1.40, more preferred 0.70≤p≤1.30. and most preferred 0.9≤p≤1.1.

The semiconductor materials can have further metallic or halfmetallic elements in amounts of naturally occurring traces. Such impurities of other metals or other semiconductor materials are typically less than 0.1 wt.-%, preferably less than 0.05 wt.-%, more preferably less than 0.005 wt.-% of the platelet semiconductor material and for the sake of clarity are not included into the formulas above.

The platelet semiconductor particles may further contain some amounts of oxygen due to surface oxidation. For example, a platelet made from pure silicon (x=0) may be oxidized on its surface. This kind of oxygen is also not included in the formulas for the sake of clarity. Preferably the platelet semiconductor particles do not contain any noticeable amount of oxygen in their interior.

The preferred embodiments of the diffractive effect pigments according to the present invention can therefore be represented as a multilayer setup A-B-A or an A-B-C system, with B being the highly reflective material and adjacent layers A and Care of a semiconductor material having a bandgap of 0.1 to 2.5 eV. If both adjacent A and C layers are present, they can be in a preferred embodiment of the same material leading to a A-B-A layer stack or they can be different (A-B-C). Preferably A and C layers are of the same material.

In further preferred embodiments, the highly reflective material is selected from the group consisting of aluminum, copper, chromium, titanium, zinc, silver, gold and alloys thereof.

In one embodiment the highly reflective material is not colored and chosen from the group of aluminum, silver, titanium, chromium and zinc and preferably of aluminum.

Here the color effects are only determined by interference effects, the diffractive structure, the degree of reflectivity of the metal core and the semiconductor layers. The thicknesses of the semiconductor layer determine the interference color.

A particular embodiment comprises aluminum as highly reflective material and as semiconductor material the alloys of the average composition of:
b. Si_{(1-y)}Sn_{y}, wherein 0<y<0.90 or
c. Ge_{(1-z)}Sn_{z}, wherein 0<z≤0.80 or
d. Si₍₁₋ₘ₋ₙ)GeₘSnₙ, wherein 0<m<1.00, 0<n<1.00 and with the proviso that m+n<1.00.

In one embodiment the highly reflective material is colored and chosen from the group of copper, gold and alloys including these materials like gold bronze (alloy of zinc and copper). Most preferred is copper.

A particular embodiment comprises copper as highly reflective material and as semiconductor material the alloys of the average composition of:
b. Si_{(1-y)}Sn_{y}, wherein 0<y<0.90 or
c. Ge_{(1-z})Sn_{z}, wherein 0<z≤0.80 or
d. Si₍₁₋ₘ₋ₙ)GeₘSnₙ, wherein 0<m<1.00, 0<n<1.00 and with the proviso that m+n<1.00.

The highly reflective metal B is a flake or platy metal material having preferably a mean primary thickness in the range from 5 to 500 nm, more preferably in the range from 5 to less than 100 nm, even more preferably in the range from 10 to less than 75 nm and most preferably in the range from 15 to 50 nm and even more preferred in a range of 20 to 40 nm.

With “primary thickness” it is meant the thickness of the respective layer material irrespective of the diffractive structure of the effect pigment (see also US 2007/0207099 A1).

For the purposes of the present invention, the primary thickness of the platy material B as well as the primary thickness of the semiconductor layers (A, C) are preferably determined by means of a scanning electron microscope (SEM). A cross section a prepared preferably by incorporating the effect pigments in a concentration of about 10 wt.-% into a two-component clearcoat (e.g. Autoclear Plus HS from Sikkens GmbH) with a sleeved brush, applied to a film with the aid of a spiral applicator (wet film thickness 26 μm), dried and cut into cross section. Using this method, the cross section of an adequate number of particles should be measured so as to realize a representative statistical evaluation. Customarily, approximately 50 to 100 particles are measured.

The average primary thickness of layers A and C can be the same or different. Typically, the average primary thickness of layers A and C can be in the range of 5-200 nm. Ideally the average primary thickness is <200nm, more ideally the average primary thickness is <100nm, and most ideally, the average primary thickness is <75nm.

A preferred mean thickness of layers A and/or C is in a range of 7 to 100 nm and more preferred in a range of 10 to 35 nm.

In further preferred embodiments the total average primary thickness of the diffractive effect pigment (meaning the sum of layers A, B and C) is in a range of 35 to below 300 nm, more preferred in a range of 50 to below 250 nm and most preferred in a range of 70 to below of 200 nm. Such effect pigments have a rather low total primary thickness leading to a high aspect ratio which favours plane-parallel orientation when the diffractive effect pigment is applied from a wet coating formulation on a substrate. A high plane-parallel orientation in turn will enhance the desired rainbow-effects. Additionally, these effect pigments have a rather high content of the central metal layer leading to a very high hiding power.

For these preferred embodiments with restricted total average thicknesses the semiconductor layers A and C preferably independently have an average thickness in the range of 7 to 60 nm, more preferably in a range of 10 to 50 nm and most preferably in a range of 10 to 35 nm.

The average thickness of the reflective layer B for these embodiments is in a range of 10 to less than 75 nm, more preferably in a range from 15 to 50 nm and most preferably in the range from 20 to 45 nm.

Surprisingly, the mechanical stability of the three-layered diffractive effect pigments is still quite high even when the total average primary thickness of the effect pigment is rather low compared to prismatic effect pigments disclosed in US 2003/0190473 A1. These effect pigments are still flexible in the sense that they align well to a substrate background when applied to a substrate.

Within the scope of the present invention, a dielectric material is an insulator (a poor electrical conductor), such as ceramics, diamond, etc., that typically has a bandgap in excess of ˜4 eV. Dielectric materials are typically optically transparent, i.e. they have very poor absorption in the visible region of the electromagnetic spectrum.

According to this invention the semiconductor material has a bandgap in the range of 0.1 to 2.5 eV and in a preferred embodiment the bandgap is in a range of 0.2 to 1.5 eV, in a more preferred embodiment the bandgap is in a range of 0.3 to 1.4 eV and in the most preferred embodiment the bandgap is in a range of 0.4 to 1.2 eV.

In a very preferred embodiment the effect the flake of a highly reflective material is made from aluminum and the semiconductor material having a bandgap of 0.1 to 2.5 eV is selected from the group consisting of germanium, alloys of germanium-silicon, and alloys of tin-silicon.

Most preferred is a diffractive effect pigment having and A-B-A layer stack, wherein the central layer B is aluminum and the adjacent semiconductor layers A are silicon or silicon-tin alloys.

### Diffractive Structure

The diffractive structure(s) of the diffractive effect pigments has or have preferably a periodic pattern with diffractive elements. The periodic pattern here refers to the smallest unit of the diffractive elements. The periodic diffractive structure preferably has 5,000 to 20,000 diffractive elements/cm, more preferably 8,000-18,000 diffractive elements/cm, and very preferably 9,000-16,000 diffractive elements/cm. Within this range, primarily visible light (about 400 to 800 nm in wavelength) is diffracted in accordance with the known principle of a diffraction lattice, with the viewer perceiving a rainbow effect as a result. It is, however, also possible for fractions of the IR radiation and/or the UV radiation to be diffracted. The periodicity substantially determines the diffracted wavelengths of the incident light. Specifically, this may be calculated in accordance with known formulae, as can be found in U.S. Pat. No. 6,692,830 B2.

Diffractive elements contemplated include, for example symmetrical triangles, asymmetrical triangles, grooves in a wide variety of different forms, rectangle functions, circles, wavy lines, cones, truncated cones, knobs, prisms, pyramids, truncated pyramids, cylinders, hemispheres, etc., and also combinations of these geometric forms and bodies.

Geometric bodies having one or more surfaces disposed parallel to the pigment surface, such as truncated cones, truncated pyramids, cylinders, or rectangle functions, for example, have a higher reflection capacity on account of these surfaces.

Geometric bodies which, relative to the pigment surface, have inclined side faces intensify the rainbow effect. Inclined side faces are side faces which, based on the substrate, have an angle of 5 to 89°, preferably of 15 to 84°, more preferably of 27 to 80°, more preferably still of 43 to 74°. Examples of suitable geometric bodies include cones, truncated cones, pyramids, truncated pyramids, etc.

In the case of truncated pyramids, there is for example a reflection at the top face parallel to the pigment surface, and an intensification of the rainbow effect at the cylindrical surface. Correspondingly, in the case of truncated pyramids, there is reflection at the top face and an intensification of the rainbow effect at the inclined side faces.

In the case of truncated cones or truncated pyramids it is of course also possible to dispose the top face not parallel to the pigment surface but instead at an incline relative to the pigment surface.

Via the geometric bodies disposed by embossing and/or shaping on the pigment surface it is possible to intensify the rainbow effect and/or the reflection capacity of the diffractive effect pigment of the invention, or to change the relative ratio of rainbow effect to reflection.

Accordingly, the geometric bodies may be present separated in sections or else mixed with one another. It is also possible of course, to dispose the geometric forms and geometric bodies with superimposition, so that, for example geometric bodies are disposed additionally on a wavy structure.

According to one further variant of the invention, unembossed or unshaped, and therefore smooth, sections may be present on the diffractive effect pigment surface, as well as embossed and/or shaped sections. By this means as well it is possible to change the relative ratio of rainbow effect to reflection.

Preferably the entire surface of the diffractive effect pigment is provided, preferably by embossing, with the diffractive structure. It is also possible, however, for only part of the diffractive effect pigment surface to be provided, preferably by embossing, with the diffractive structure, and/or to be shaped to form a diffractive structure. It is preferred for at least 60%, more preferably at least 75%, and very preferably at least 90% of the diffractive effect pigment surface to be embossed with a diffractive structure or shaped to form a diffractive structure.

In one particularly preferred embodiment, the diffractive structure comprises or consists of wavy—for example, sinusoidal—lines, cones, or truncated cones. In an especially preferred embodiment, the diffractive structure comprises or consists of sinusoidal lines, since first this form is particularly easy to emboss and secondly it produces a very strong diffraction effect. In the case of these sinusoidal lines, the diffractive structure is preferably embossed on the whole metallic effect pigment.

In order to be able to obtain a distinctly perceptible effect, the diffractive structure preferably has a certain minimum depth, since otherwise it is not possible adequately to develop the physical effect of diffraction. The diffractive structure ought therefore preferably to have a depth (measured as “peak to valley”, see also FIG. 1 and of US 2007/0207099 A1) of at least 40 nm, preferably 40 nm to 600 nm, and more preferably of 50 nm to 400 nm, and very preferably of 100 nm to 250 nm. Above 600 nm, the structure as a whole may no longer have stability.

The depth of the diffractive structure may therefore exceed the average primary layer thickness of the whole effect pigment. This is preferably the case especially when the entire effect pigment is shaped to form a diffractive structure.

Regarding the sizes and size distributions of the diffractive flaky effect pigments typical size ranges of printing inks or cosmetic formulations are chosen. Preferably the flaky effect pigment have a d₅₀ of the particle size distribution is in a range of 2 to 100 μm, more preferably in a range of 5 to 45 μm, furthermore preferred in a range of 6 to 35 μm and most preferably in a range of 7 to 30 μm.

The pigment size is typically indicated using quantiles (d values) from the volume averaged particle size distribution. Here, the number indicates the percentage of particles smaller than a specified size contained in a volume-averaged particle size distribution. For example, the d₅₀ value indicates the size where 50% of the particles are smaller than this value. These measurements are conducted e.g. by means of laser granulometry using a particle size analyzer manufactured by Horiba and is a Horiba LA 950 instrument. The measurements are conducted using Fraunhofer approximation and suitable parameters according to information from the manufacturer.

The d₁₀-values characterize the relative amount of fine particles and typically range from 2 to 20 μm and preferably from 4 to 15 μm.

The d₉₀-values characterize the relative amount of coarse particles and typically range from 15 μm to 140 μm and preferably from 20 μm to 50 μm.

The width of the particle size distribution can be characterized by the span defined as (d₉₀-d₁₀)/d₅₀ and preferably this span is in a range of 1.50 to 2.2 and more preferably in a range of 1.6 to 2.0.

### Effect Pigment Encapsulation

In certain embodiments the effect pigments might be encapsulated with a further layer. Such encapsulation might be necessary to ensure gassing stability for water-based coating systems or water-based printing inks, for example. As the edges of the flaky metal are not covered by the semiconductor layer the effect pigment can be particularly attacked by a corrosive media such as a water-based coating formulation.

Typically, these encapsulating layers will be used in an amount of and from a materials which effectively protect the effect pigment from corrosion but at the same time do not alter the optical appearance too much. Typically, materials of low refractive index are used and typically the materials are not colored or when they are colored, they are used in very small amounts. Therefore, the enveloping layer is of an essentially optical non-active material. Preferably, the optical thickness (product of geometrical layer thickness and refractive index) of the enveloping layer is less than 34 nm and more preferably less than 30 nm. Herein, the refractive index denotes to literature values averaged for the visible range (wavelengths 400 to 700 nm) for the respective materials and not to the effective refractive index of the layer.

In preferred embodiments the further layer encapsulates essentially the whole diffractive effect pigment and consists of a layer of Mo-oxide, SiO₂, Al₂O₃, or surface modifiers like organofunctional silanes, phosphate ester, phosphonate esters, phosphite esters and combinations thereof.

More preferably the further layer encapsulates the whole diffractive effect pigment and consists of a layer of Mo-oxide, SiO₂ and optionally a surface modifier like organofunctional silanes. Such systems are described e.g. in WO 2019/110490 A1. In another preferred embodiment the optically non-active layer consists of a layer of SiO₂ and optionally a further layer of organofunctional silanes which serve as modifier agents of the SiO₂ surface.

The organofunctional silanes are primarily needed as surface modifiers here to adjust the chemical compatibility of the diffractive effect pigment to the binder medium of the final application as described in e.g. EP 1084198 A1.

The organofunctional silanes used preferably as surface modifiers, which contain suitable functional groups, are available commercially and are produced, for example, by Evonik, Rheinfelden, Germany and sold under the trade name “Dynasylan®”. Further products can be purchased from OSi Specialties (Silquest® silanes) or from Wacker (Genosil® silanes).

Examples of suitable organofunctional silanes are 3-methacryloxypropyl trimethoxy silane (Dynasylan MEMO), vinyl tri(m)ethoxy silane (Dynasylan VTMO or VTEO), 3-mercaptopropyl tri(m)ethoxy silane (Dynasylan MTMO or 3201), 3-glycidyloxypropyl trimethoxy silane (Dynasylan GLYMO), tris(3-trimethoxysilylpropyl) isocyanurate (Silquest Y-11597), gamma-mercaptopropyl trimethoxy silane (Silquest A-189), bis(3-triethoxysilylpropyl) polysulfide (Silquest A-1289), bis(3-triethoxysilyl) disulfide (Silquest A-1589), beta(3,4-epoxycyclohexyl) ethyl trimethoxysilane (Silquest A-186), gamma-isocyanatopropyl trimethoxsilane (Silquest A-Link 35, Genosil GF40), (methacryloyloxymethyl) trimethoxysilane (Genosil XL 33) and (isocyanatomethyl) trimethoxysilane (Genosil XL 43).

In one preferred embodiment the organofunctional silane mixture that modifies the SiO₂ layer comprises at least one amino-functional silane. The amino function is a functional group which is able to enter into chemical interactions with the majority of groups present in binders. This interaction may involve a covalent bond, such as with isocyanate or carboxylate functions of the binder, for example, or hydrogen bonds such as with OH or COOR functions, or else ionic interactions. It is therefore very highly suitable for the purpose of the chemical attachment of the diffractive effect pigment to different kinds of binder.

The following compounds are employed preferably for this purpose: aminopropyl trimethoxy silane (Dynasylan AMMO), aminopropyl triethoxy silane (Dynasylan AMEO), N-(2-aminoethyl)-3-aminopropyl trimethoxy silane (Dynasylan DAMO), N-(2-aminoethyl)-3-aminopropyl triethoxy silane, triamino-functional trimethoxy silane (Silquest A-1130), bis(gamma-trimethoxysilylpropyl)amine (Silquest A-1170), N-ethyl-gamma-aminoisobutyl trimethoxy silane (Silquest A-Link 15), N-phenyl-gamma-diaminopropyl trimethoxy silane (Silquest Y-9669), 4-amino-3,3-dimethylbutyltrimethoxy-silane (Silquest Y-11637). (N-cyclohexylaminomethyl)-triethoxy silane (Genosil XL 926), (N-phenylaminomethyl)-trimethoxy silane (Genosil XL 973) and mixtures thereof. In another embodiment pre-hydrolysed and pre-condensated organofunctional silanes may be used as described in EP 3080209 B1.

In other embodiments the encapsulating optically non-active layer is based on SiO₂ which is further modified by organofunctional silanes to produce a hybrid inorganic/organic layer. Such modifying organofunctional silanes may be a diphenyl dimethoxy silane, a diphenyl diethoxy silane, an amino silane and the like. In other embodiments the encapsulating optically non-active layer is based on SiO₂ as inorganic component and layers comprising organic oligomers or polymers are formed as described in WO 2007/017195 A2 or in WO 2016/120015 A1.

In other embodiments the organofunctional silanes or other corrosion inhibitors like phosphate ester, phosphonate esters, phosphite esters and combinations thereof may be coated directly on the diffractive effect pigment to impart corrosion and gassing stability especially to the edges of the effect pigment, which are not coated by the semiconductor layer.

### Method of Manufacture

The effect pigments according to the present invention can be manufactured using a physical vapor deposition (PVD) process.

A method of manufacturing the diffractive effect pigment using a PVD process comprises the steps:
(a1) applying a release layer to a linearly movable flexible substrate,
(a2) introducing a diffractive structure into the release layer, preferably by embossing, and
(a3) depositing a first semiconductor layer onto the release layer on the linearly movable flexible substrate, in a vacuum chamber having a vapor-deposition section, by means of physical vapor deposition (PVD), and
(a4) depositing a metal layer onto the first semiconductor layer on the linearly movable flexible substrate, in a vacuum chamber having a vapor-deposition section, by means of physical vapor deposition
(a5) depositing a second semiconductor layer onto the metal layer on the linearly movable flexible substrate, in a vacuum chamber having a vapor-deposition section, by means of physical vapor deposition
   or
(b1) applying a release layer to a linearly movable flexible substrate,
(b2) depositing a first semiconductor layer onto the release layer on the linearly movable flexible substrate, in a vacuum chamber having a vapor-deposition section, by means of physical vapor deposition (PVD), and
(b3) depositing a metal layer onto the first semiconductor layer on the linearly movable flexible substrate, in a vacuum chamber having a vapor-deposition section, by means of physical vapor deposition
(b4) depositing a second semiconductor layer onto the metal layer on the linearly movable flexible substrate, in a vacuum chamber having a vapor-deposition section, by means of physical vapor deposition
(b5) introducing a diffractive structure onto and/or into any of the structures of step (b2) to (b4), preferably by embossing,
c) stripping the material stack from the flexible substrate in a solvent and
d) optionally further steps including particle sizing, particle classification and solvent exchange and/or solvent dispersion.

The first method denoted with the (a1) to (a5) features is clearly preferred due to its ease manufacturing. Effect pigments made by this method adopt the diffractive structure within the whole 3-layered effect pigment.

The steps (a1), (a2), (b1), (c) and (d) can be done in conventional manner as disclosed e.g. in U.S. Pat. Nos. 6,200,399 B1, 5,672,410 A, 5,629,068 A, 5,624,076 A, 6,068,691 A, and WO 1993/023481 A1.

The flexible substrate is usually a web made from polymers and most preferably a PET polymer.

The semiconductor materials preferably have an average atomic composition of:
a. Si₍₁₋ₓ₎Geₓ, wherein 0≤x≤1.00 or
b. Si_{(1-y)}Sn_{y}, wherein 0<y<0.90 or
c. Ge_{(1-z)}Sn_{z}, wherein 0<z≤0.80 or
d. Si₍₁₋ₘ₋ₙ₎GeₘSnₙ, wherein 0<m<1.00, 0<n<1.00 and with the proviso that m+n<1.00 or
e. SiOₚ, wherein 0.30≤p≤1.50.

In steps (a3), (a5), (b2) and (b4) in one embodiment semiconductor alloys of a predetermined composition are independently used as bulk materials which are evaporated by suitable means to produce respective gas molecules which are transferred to the flexible substrate coated with a release layer under ultra-high vacuum conditions. In another embodiment two or three suitable bulk semiconductor materials of a predetermined purity are used wherein their vapor clouds are allowed to overlap before reaching the substrate.

These steps as well as steps (a4) or (b3) can be conducted as an electron beam process, magneton sputtering, resistive evaporation or inductive heating. Most preferred is evaporation of the semiconductor bulk material by an electron beam process.

The thicknesses of the deposited layers can be controlled by optical transmission sensors measuring the optical density (OD) which is known to correlate to the layer thickness.

In further preferred embodiments the total average primary thickness of the diffractive effect pigment (meaning the sum of layers A, B and C) is in a range of 35 to below 300 nm, more preferred in a range of 50 to below 250 nm and most preferred in a range of 70 to below of 200 nm. Such effect pigments have a rather low total primary thickness leading to a high aspect ratio which favours plane-parallel orientation when the diffractive effect pigment is applied from a wet coating formulation on a substrate. A high plane-parallel orientation in turn will enhance the desired rainbow-effects. Additionally, these effect pigments have a rather high content of the central metal layer leading to a very high hiding power.

For these preferred embodiments with restricted total average thicknesses the semiconductor layers A and C preferably independently have an average thickness in the range of 7 to 60 nm, more preferably in a range of 10 to 50 nm and most preferably in a range of 10 to 35 nm.

The average thickness of the reflective layer B for these embodiments is in a range of 10 to less than 75 nm, more preferably in a range from 15 to 50 nm and most preferably in the range from 20 to 45 nm.

The release coat step may be skipped if a metallized film is to be produced without intention of stripping the stack material. Additionally, steps (a5) and (b4) may be skipped, if a 2-layered film is to be produced.

The above process produces a material stack that may be stripped from the flexible substrate in subsequent step c). The kind of release coats used here and the solvents for stripping the film stack are well known in the art. Examples for the release-coats are acrylics, methacrylics or polystyrol. They can be also other organic materials as described in US 2004/0131776 A1 or in US 20100062244 A1. Preferred solvents for stripping of the material stack from the flexible substrate in step c) are acetone, ethyl acetate, propylene glycol methoxy ether, isopropyl alcohol, ethanol, or water. The steps of depositing the semiconductor and metal films may be mirrored on the opposing side of the release agent coated flexible substrate and multiple stacks may be deposited on a single flexible substrate by repeating the process. Additionally, a single side may be coloured with the opposing side maintaining the metal optical properties by removing one of the semiconductor layers. If the first semiconductor layer is removed, the metal side will be coloured, while if the second semiconductor layer is removed, the web-side will be coloured.

In the case of pigment manufacturing, the material deposited from the abovementioned substrate is typically stripped utilizing a solvent or mechanical stripping process, followed by post processing steps, which may include particle sizing, particle classification, and solvent dispersion.

The rather thin three-layered prismatic effect pigments are easy to process during manufacturing, especially during the step of particle sizing.

In preferred embodiments highly reflective metal film has a thickness in a range of 5 to 500 nm.

In further preferred embodiments the first semiconductor layer and the second semiconductor layer are composed of the same material.

In further preferred embodiments the first and the second semiconductor layers essentially have the same thickness. With “essentially the same thickness” it meant that the deviations of the thickness of the first and the second semiconductor layers are less than 15%. If additionally the first and second semiconductor layer are made from the same material the colouration on will be the same on both sides of the reflective metal.

### Applications

The colour and other optical properties of the diffractive effect pigment according to the present invention can be made visible and measurable by incorporating the diffractive effect pigment in a colourless binder system and by using the obtained composition to coat a substrate. For example, an ink-composition can be obtained by mixing about 6 wt. % of the diffractive effect pigment according to the present invention with a colourless nitrocellulose binder and preparing a drawdown on a sample card, for example a BYK Gardner drawdown card.

The effect pigments according to the present invention can be used of a broad range of applications, typically for metallic effect pigments, such as coatings, inks, cosmetics or plastics.

Coating or ink compositions comprising these effect pigments can show a distinct reflection color, a rainbow effect which can also overlap with a color flop effect. In certain embodiments some color regions like the blue or the red region of the rainbow are muted.

Further embodiments of this invention are directed to the use of the flaky diffractive effect pigment as described above, including all preferred embodiments described therein in coatings, printing inks, cosmetic formulations or plastics. Particularly preferred is the use of the flaky diffractive effect pigment in cosmetic formulations and security printing inks.

Further embodiments of this invention are directed a coating composition, an ink composition or a cosmetic composition comprising a diffractive effect pigment as described above, including all preferred embodiments described therein. Particularly preferred are cosmetic formulations and security printing inks containing the diffractive effect pigments.

### Further Aspects

This invention includes further aspects which are directed to the films which are preferably produced in a PVD chamber prior to particle sizing. In these films also two-layered materials may be of interest.

A first aspect is directed to a film, wherein the film has a diffractive structure and consists of a first semiconducting material having a bandgap of 0.1 to 2.5 eV and coated thereon a layer of a highly reflective material.

A second aspect is directed to a film according to the first aspect, wherein according to the first aspect, wherein a second layer of a semiconducting material having a bandgap of 0.1 to 2.5 eV is coated onto the layer of a highly reflective material, wherein this second semiconductor layer is independent of the first semiconductor layer with respect to its composition and thickness.

A third aspect is directed to any of the previous aspects, wherein the highly reflective material is selected from the group consisting of aluminum, copper, chromium, titanium, zinc, silver, gold and alloys thereof.

A fourth aspect is directed to any of the previous aspects, wherein the first and independently the second semiconductor layers have an average atomic composition:
a. Si₍₁₋ₓ₎Geₓ, wherein 0≤x≤1.00 or
b. Si_{(1-y)}Sn_{y}, wherein 0<y<0.90 or
c. Ge_{(1-z)}Sn_{z}, wherein 0<z≤0.80 or
d. Si₍₁₋ₘ₋ₙ₎GeₘSnₙ, wherein 0<m<1.00, 0<n<1.00 and with the proviso that m+n<1.00 or
e. SiOₚ, wherein 0.30≤p≤1.50.

A fifth aspect is directed to any of the previous aspects, wherein the flake of a highly reflective material has an average primary thickness in the range from 5 to 500 nm and preferably in a range of 5 to less than 100 nm.

A sixth aspect is directed to any of the previous aspects, wherein the layers of the semiconductor materials have an average primary thickness in the range from 5 to 200 nm and preferably in a range of 10 to 100 nm.

A seventh aspect is directed to any of the previous aspects, wherein the total average primary thickness of the three layers (meaning the sum of layers A, B and C) is in a range of 35 to below 300 nm, more preferred in a range of 50 to below 250 nm and most preferred in a range of 70 to below of 200 nm.

All other aspects relating to the compositions or thicknesses of the materials of the two- or three-layered film stack are the same as previously disclosed in this invention for the effect pigments.

### EXAMPLES

### Examples 1: 3-Layer Material (Si—Al—Si)

3-layer materials were deposited onto a film coated with a releasing agent which was embossed with a specified prismatic pattern. The ebeam source accelerating voltage was held at a constant 10 kV throughout the runs. The ebeam source was positioned 36 cm below the film during deposition. In a first step a Si-layer was deposited on a clear polyester film with an embossed release coat layer using PVD ebeam evaporation. Ebeam current was set at the beginning of the run and web speed was utilized to manipulate the silicon layer thicknesses. In a next step an Al layer was deposited corresponding to approximately 1.2-1.7 OD (optical density). In a third process step, a further layer of Si was deposited using the same parameters as the first step. Again, ebeam current was set at the beginning of the run, and web speed was utilized to manipulate the silicon layer thicknesses. For all depositions, optical transmission sensors in combination with current adjustment was utilized to target appropriate layer thickness. The thickness of the 2 silicon layers was targeted to be the same, so that the web side and metal side of each condition would be the same colour. Yellow and orange colouration with prismatic effect were successfully produced. The average colouration of the web and metal side of the films matched well in the material set. The films displayed primary yellow or orange coloration with unique colour flop effects. The yellow film colour flops from red, orange, yellow, green, highly muted blue, and extremely muted violet. The orange film colour flops from red, orange, yellow, green, extremely muted blue, and extremely muted violet.

The deposited materials from Example 1 were stripped from the polyester film and homogenized to a particles size of approximately 30 μm (d₅₀ value). The average particle thickness obtained via SEM analysis is 60+/−6 nm and 75+/−13 nm for the yellow and orange pigments, respectively. The contrast between Si and Al in the SEM was not adequate to distinguish individual layers. Pigments of yellow and orange colouration were prepared in ethanol with 8.2 wt. % and 10.8 wt. % non-volatile content (NVM), respectively. Inks were prepared in a binder system, composed of Hagedorn H7 Nitrocellulose binder (obtainable from Hagedorn AG, Osnabrück, Germany) in a solvent blend of ethyl acetate and propylene glycol monomethyl ether. Formulations were based on weight ratios of metals content to binder, with binder-to-pigment weight ratios of 58:42 and 51:49 for the yellow and orange pigments, respectively. The samples were drawn down on a flat Leneta drawdown cards and polyester film for reverse printing observation with a wire wound rod at 60 μm wet film thickness. Since quantitative characterization of diffractive effect pigments are challenging, the pigments will be described in a qualitative manner.

As Comparative Example 1 a standard prismatic aluminum pigment, such as Eckart's Metalure Prismatic H50700 AE shows when applied under similar conditions a diffractive pattern in the order: red, orange, yellow, green, blue, violet. Each colour is distinct, and vibrant. The yellow and orange 3-layer prismatic pigments, however, display heavily muted coloration flops of certain wavelength ranges. The yellow drawdowns colour flop in the order: red, orange, yellow, green, highly muted blue, and extremely muted violet. The orange drawdowns colour flop in the order: red, orange, yellow, green, extremely muted blue, and extremely muted violet.

### Pre-Examples 2: 3-Layer Material (Ge—Al—Ge)

A 3-layer material was deposited onto a film coated with a releasing agent which was embossed with a specified prismatic pattern. The ebeam source accelerating voltage was held at a constant 10 kV throughout the runs. The ebeam source was positioned 36 cm below the film during deposition. In a first step a Ge-layer was deposited on a clear polyester film with an embossed release coat layer using PVD ebeam evaporation. Ebeam current was set at the beginning of the run and web speed was utilized to manipulate the germanium layer thicknesses. In a next step an Al layer was deposited corresponding to approximately 1.6-2.1 OD. In a third process step, a further layer of Ge was deposited using the same parameters as the first step. Again, ebeam current was set at the beginning of the run, and web speed was utilized to manipulate the germanium layer thicknesses. For all depositions, optical transmission sensors in combination with current adjustment was utilized to target appropriate layer thickness. The thickness of the two germanium layers was targeted to be the same, so that the web side and metal side of each condition would be the same colour. The average colouration of the web and metal side of the films matched well in the material set. The film displayed primary teal blue coloration with unique colour flop effects. The teal blue film colour flops from extremely muted red, highly muted orange, yellow, green, blue, and violet.

The deposited material from Example 2 was stripped from the polyester film and homogenized to a particles size of approximately 30 μm (d₅₀ value). The average particle thickness obtained via SEM analysis is 80+/−3 nm for the blue pigments. Individual pigment layers were distinguishable at 28+/−3 nm and 25+/−2 nm for germanium and aluminum layers, respectively. Pigments were prepared with a 15 wt. % non-volatile content (NVM) in ethanol. Inks were prepared in a binder system, composed of Hagedorn H7 Nitrocellulose binder (obtainable from Hagedorn AG, Osnabrück, Germany) in a solvent blend of ethyl acetate and propylene glycol monomethyl ether. Formulations were based on weight ratios of metals content to binder, with a binder-to-pigment weight ratio of 43:57. The samples were drawn down on a flat Leneta drawdown cards and polyester film for reverse printing observation with a wire wound rod at 60 μm wet film thickness. Since quantitative characterization of diffractive effect pigments are challenging, the pigments will be described in a qualitative manner.

As described above the Comparative Example 1 standard prismatic aluminum pigment shows a color flop in the order: red, orange, yellow, green, blue, violet. Each colour is distinct, and vibrant. The real blue 3-layer prismatic pigments of the inventive Example 2, however, display heavily muted coloration flops of certain wavelength ranges. The blue drawdowns colour flop in the order: extremely muted red, highly muted orange, muted yellow, green, blue, and violet.

1. A flaky diffractive effect pigment having a diffractive structure and a 3-layer stack consisting of a core of a flake of a highly reflective material having a first major interface and opposed to this first interface a second major interface, and at least one side surface and directly adjacent on both of these major interfaces a layer of a semiconducting material having a bandgap of 0.1 to 2.5 eV, wherein the layers of the semiconductor material have an average primary thickness in the range from 10 to 100 nm and wherein the total primary thickness of the effect pigment is in a range of 35 to below 300 nm.

2. The flaky diffractive effect pigment according to claim 1, wherein the highly reflective material is selected from the group consisting of aluminum, copper, chromium, titanium, zinc, silver, gold and alloys thereof.

3. A flaky diffractive effect pigment according to claim 1, wherein the layers of semiconductor material independently comprise a semiconductor material having an average atomic composition according to one of the following:
a. Si(₁₋ₓ)Geₓ, wherein 0≤x≤1.00 or
b. Si_{(1-y)}Sn_{y}, wherein 0<y<0.90 or
c. Ge_{(1-z)}Sn_{z}, wherein 0<z≤0.80 or
d. Si₍₁₋ₘ₋ₙ₎GeₘSnₙ, wherein 0<m<1.00, 0<n<1.00 and with the proviso that m+n<1.00 or
e. SiOₚ, wherein 0.30≤p≤1.50.

4. A flaky diffractive effect pigment according to claim 1, wherein the semiconductor material has an average atomic composition of:
a. Si₍₁₋ₓ₎Geₓ, wherein 0.02<x≤0.9 and preferably 0.04≤x≤0.8 or
b. Si_{(1-y)}Sn_{y}, wherein 0.02≤y≤0.75 or
c. Ge_{(1-z)}Sn_{z}, wherein 0.02≤z≤0.60 or
d. Si₍₁₋ₘ₋ₙ₎GeₘSnₙ, wherein 0.02≤m≤0.8, 0.02≤n≤0.75 and with the proviso that m+n<1.00 or
e. SiOₚ, wherein 0.50≤p≤1.40.

5. A flaky diffractive effect pigment according to any of the preceding claims claim 1, wherein the semiconductor material has an average atomic composition of:
a. Si₍₁₋ₓ₎Geₓ, wherein 0.10≤x≤0.65 or
b. Si_{(1-y)}Sn_{y}, wherein 0.05≤y≤0.55 or
c. Ge_{(1-z)}Sn_{z}, wherein 0.05≤z≤0.50 or
d. Si₍₁₋ₘ₋ₙ₎GeₘSnₙ, wherein 0.05≤m≤0.65, 0.05≤n≤0.55 and with the proviso that m+n<1.00 or
e. SiOₚ, wherein 0.70≤p≤1.30.

6. The flaky diffractive effect pigment according to claim 1, wherein the flake of a highly reflective material has an average primary thickness in the range from 5 to less than 100 nm.

7. The flaky diffractive effect pigment according to claim 1, wherein the highly reflective material is not colored and comprises aluminum, titanium, chromium or zinc.

8. The flaky diffractive effect pigment according to claim 1, wherein the diffractive effect pigment is further encapsulated with an outer layer.

9. The flaky diffractive effect pigment according to claim 1, wherein the at least one diffractive structure has at least one periodic pattern with diffractive elements comprising geometric shapes or bodies.

10. The flaky diffractive effect pigment of claim 9, wherein the periodic pattern has 5,000 to 20,000 diffractive elements/cm.

11. The flaky diffractive effect pigment of claim 9, wherein the diffractive structure has a depth of at least 40 nm.

12. A method of making a flaky diffractive effect pigment using a PVD process comprising:
(a1) applying a release layer to a linearly movable flexible substrate,
(a2) introducing a diffractive structure into the release layer, preferably by embossing, and
(a3) depositing a first semiconductor layer onto the release layer on the linearly movable flexible substrate, in a vacuum chamber having a vapor-deposition section, by means of physical vapor deposition (PVD), and
(a4) depositing a metal layer onto the first semiconductor layer on the linearly movable flexible substrate, in a vacuum chamber having a vapor-deposition section, by means of physical vapor deposition
(a5) depositing a second semiconductor layer onto the metal layer on the linearly movable flexible substrate, in a vacuum chamber having a vapor-deposition section, by means of physical vapor deposition
or
(b1) applying a release layer to a linearly movable flexible substrate,
(b2) depositing a first semiconductor layer onto the release layer on the linearly movable flexible substrate, in a vacuum chamber having a vapor-deposition section, by means of physical vapor deposition (PVD), and
(b3) depositing a metal layer onto the first semiconductor layer on the linearly movable flexible substrate, in a vacuum chamber having a vapor-deposition section, by means of physical vapor deposition
(b4) depositing a second semiconductor layer onto the metal layer on the linearly movable flexible substrate, in a vacuum chamber having a vapor-deposition section, by means of physical vapor deposition
(b5) introducing a diffractive structure onto and/or into any of the structures of step (b2) to (b4), and
c) stripping the material stack from the flexible substrate in a solvent,
the resulting flaky diffractive effect pigment having a diffractive structure and a 3-layer stack consisting of a core of a flake of a highly reflective material having a first major interface and opposed to this first interface a second major interface, and at least one side surface and directly adjacent on both of these major interfaces a layer of a semiconducting material having a bandgap of 0.1 to 2.5 eV, wherein the layers of the semiconductor material have an average primary thickness in the range from 10 to 100 nm and wherein the total primary thickness of the effect pigment is in a range of 35 to below 300 nm.

13. The method of manufacturing according to claim 12, wherein the reflective metal has an average primary thickness in a range of 5 to less than 100 nm.

14. The method of manufacturing according to claim 12, wherein the first semiconductor layer and the second semiconductor layer are composed of the same material.

15-16. (canceled)

17. The flaky diffractive effect pigment according to claim 7, wherein the highly reflective material comprises aluminum.

18. The method of manufacturing according to claim 12, comprising steps (b1)-(b5), wherein step (b5) comprises embossing.

19. The method of claim 12, further comprising one or more steps of particle sizing, particle classification and solvent dispersion.

20. An ink composition comprising the effect pigment of claim 1 and a binder.

21. A product comprising the effect pigment of claim 1 in the form of a cosmetic composition.
